(19) United States
(12) Patent Application Publication
(10) Pub. No.: US 2005/0271600 A1
(43) Pub. Date: Dec. 8, 2005

(54) Fiber supplement

(76) Inventors: Henry D. Coleman, Hastings, NY (US); R. Neil Sudol, Scarsdale, NY (US); William J. Sapone, Southport, CT (US)

Correspondence Address: WILLIAM J. SAPONE;COLEMAN SUDOL SAPONE P.C., 714 COLORADO AVENUE, BRIDGE PORT, CT, 06605, US

(21) Appl. No.: 11/144,090

(22) Filed: Jun. 2, 2005

Related U.S. Application Data
(60) Provisional application No. 60/576,529, filed on Jun. 3, 2004.

(51) Int. Cl. A61K 35/78; A61K 33/32; A61K 31/385; A61K 31/716

(52) U.S. Cl. 424/48; 424/440; 424/641; 424/745; 424/752; 514/57; 514/27; 514/18; 514/440; 514/456; 424/776

(57) ABSTRACT

A fiber supplement, preferably formulated with a confectionery base, removes or prevents the bio-accumulation of heavy metals in the body. The supplement has one or more natural fibers that bind any heavy metal released or found in the intestinal tract for removal by excretion. Using the fiber supplement limits the accumulation of heavy metals in the body, promotes removal of heavy metals previously accumulated in the body and thereby alleviates the symptoms and conditions associated with heavy metal toxicity.

### CROSS REFERENCE TO RELATED APPLICATIONS

This application claims priority in U.S. Provisional Patent application No. 60/576,529 filed Jun. 3, 2004.

### BACKGROUND

This invention is directed to a fiber supplement. More particularly, this invention is directed to a fiber supplement for assisting the body in cleansing itself of undesirable metals.

Mercury has been implicated in a vast array of disorders and diseases, from vascular disease to immunological malfunctions, from renal dysfunction to autism and related neurological disorders such as attention deficit disorder. Lead, arsenic and cadmium are also known to be toxic in any substantial quantities.

High levels of heavy metals such as mercury and lead are most common in people who have been exposed to high concentrations of the metals, for example, those individuals who have had the misfortune of living in a toxic waste area or near a chemical processing plant. However, even people exposed to small concentrations of a heavy metal can, over time, accumulate dangerous levels of the substance. This long term exposure has evidently occurred in children who received a series of vaccinations preserved with a mercury containing compound. Although no single vaccination likely contained enough mercury to cause any damage, the accumulation of mercury from multiple vaccinations over a two or three year period resulted in dangerous levels of mercury in a significant percentage of children.

It is known that mercury toxicity can disrupt the immune system, and may be implicated in auto-immune disease, and many food and environmental allergies may be attributed to sensitivity caused by mercury exposure. It is also known that the effects of mercury in the body can include behavioral changes, depression, confusion, irritability and hyperactivity, as well as fatigue, insomnia, slurred speech, etc.

It does not appear to be a coincidence that these symptoms correlate with many childhood conditions that are approaching near epidemic proportions. For example, Attention Deficit Disorder (ADD) and Attention Deficit/Hyperactivity Disorder (ADHD) (severally and collectively hereinafter referred to as “AD”) are developmental disorders of self-control. They consist of problems with attention span, impulse control and activity level. These problems are reflected in impairment of a person's will or capacity to control his or her own behavior relative to the passage of time and to keep future goals and consequences in mind. The number of children having AD and related conditions, as well as allergies, appears to have grown in parallel with the level of mercury exposure through vaccines and otherwise. At present there are various drug treatments used to address these conditions, but these are directed to alleviating symptoms and are not directed to removing a possible source of the condition.

On the cellular level, mercury appears to inhibit the natural action of enzyme systems, to depolarize cell membranes, to increase intracellular calcium, to alter neurotransmitter release and inhibition. Impact on the neurological system, digestive tract and immune system are implicated.

Virtually everyone who lives in today's society, consuming modern goods and living in an environment polluted with decades of heavy industrial output, can be expected to take in significant quantities of heavy metals over a lifetime. Mercury can leach out of dental fillings, at least when the fillings are first created, are worked on by a dentist or are subject to considerable fatigue stressing. Aluminum, a lighter but still highly reactive metal to be sure, but not a mineral found in natural organic tissues, can be absorbed from aluminum in cans, foils, cookware and cosmetics such as antiperspirants. Long term intake of even trace quantities of heavy metals can produce or exacerbate debilitating illness, especially neurological disease states and conditions. Where the heavy metals are absorbed in the central nervous system, various neural diseases including neuro-degenerative diseases may result, including, for instance, Alzheimer's, Parkinson's and related diseases. In children, heavy metal toxicity (especially lead and mercury, but also cadmium and arsenic, among others) is implicated in speech impediments, learning disabilities, attention deficit disorder (ADD) and attention deficit hyperactivity disorder (ADHD), autism, autism spectrum diseases and related developmental diseases.

There are medical treatments for acute heavy metal toxicity, relying on the use of strong chelating agents such as DMSA or EDTA. However, such treatments are not available to those exhibiting less dramatic but more insidious effects of low levels of mercury, lead and other heavy metal toxicity caused by bio-accumulation, and such medical treatments cannot be used as a prophylaxis to limit bio-absorption of heavy metals or to support the natural bodily processes for sequestering and removing heavy metal contaminants.

Any composition or method that provides a means to reduce heavy metal levels, using natural ingredients such as specific nutritional or dietary supplements, would be quite useful, particularly for those who do not need therapeutic intervention but rather who wish to actively take steps to maintain their health before such intervention is called for.

Accordingly, people who wish to protect themselves from the risks associated with exposure to heavy metals by enhancing removal of these materials from the body have a need for products to assist the body in dealing with such heavy metals and for supporting the body's natural restorative functions.

### OBJECTS OF THE INVENTION

An object of the present invention is to provide a fiber supplement for assisting the body in performing natural cleansing or detoxification functions.

A more specific object of the present invention is to provide a fiber supplement for use in assisting the body in the removal of heavy metals such as nickel, lead, mercury, arsenic, cadmium, aluminum and tin, and also iron and copper.

Another object of the present invention is to provide such a fiber supplement which assists in not only removing heavy metals from the body but also assists in reversing the effects of heavy metal toxicity.

It is still another object of the present invention to provide a method for reducing or removing metals in individuals who may be at risk for or show symptoms of heavy metal toxicity which may manifest as Alzheimer's disease, Parkinson's disease, learning disabilities, autism, ADD, ADHD, speech disabilities and related neurological conditions, as well as chronic fatigue syndrome, sleeplessness, depression, anxiety, bipolar disease, multiple sclerosis, allergies, cardiovascular disease and other diseases and conditions where heavy metal toxicity in a patient may be implicated.

These and other objects of the present invention will be apparent from the description herein. Although every object is attained by at least one embodiment of the invention, there is not necessarily any embodiment in which all of the objects of the invention are achieved.

### SUMMARY OF THE INVENTION

The present invention is principally directed to a fiber supplement to be taken regularly, preferably at least once daily and more preferably at least twice daily for purposes of assisting natural cleansing mechanisms to remove heavy metals from the body, particularly from the intestinal tract. The fiber supplement may be taken directly as a supplement, or be used as a food additive, thereby administered as part of typical food or beverage consumption.

Fiber supplements in accordance with the present invention are intended for long term use and are administrable from once up to about six or more times daily for bolstering the body's natural defenses against heavy metals. The fiber supplement is preferably a blend of dietary fibers integrated with a carrier and administered at least twice daily. The amounts of the various fiber components are relatively small and fall within acceptable limits which are now included individually in a number of products unrelated to the present invention. When used on a consistent/continual basis, the fiber supplement serves to assist the body's natural mechanisms for the removal of toxic metals by binding these metals in the intestinal tract for removal by excretion. The fiber supplement is particularly effective for the removal of mercury and lead. The supplements are also at least partially effective for assisting in the removal of other heavy metals such as nickel, cadmium, aluminum, arsenic, and tin, as well as iron and copper, when desirable.

A fiber supplement in accordance with an embodiment of the present invention includes at least one dietary fiber from the group consisting of pectin, preferably apple pectin, beet pectin, water soluble and water insoluble bran fiber, preferably water soluble what or oat bran fiber and water insoluble wheat or oat bran fiber, or combinations thereof, as a metal binding fiber or fiber blend, optionally mixed with a bulk fiber selected from the group consisting of inulin, psyllium, cellulose fibers, among others, and combinations thereof. Various natural body processes assist in the removal of heavy metals from the vascular and central nervous systems, but then these must be removed via an excretion pathway, and often, if not bound and removed, there is the possibility for re-uptake or recycle of the heavy metal. The fiber supplement of the invention assures the capture and removal via fecal excretion of any heavy metals disposed in or delivered to the intestinal tract.

The fiber supplement preferably has fiber present in an amount sufficient to capture heavy metals in the intestinal tract to effectively prevent recycling of the heavy metal species back into the vasculature system. Preferably, the metal binding fibers trap these metals, and the bulk fiber assures their removal via excretion. Of course, some recycling may occur, but the fiber supplement reduces or prevents recycling of all of the captured metal ions and thus enables the body to cleanse itself of the target heavy metal. Through long term use of the inventive composition, substantial progress can be made in eliminating such heavy metals from the body, which also thereby functions as a prophylactic to capture such heavy metal species as are encountered through normal environmental interaction, such as present in trace amounts in food or water and air, so as to prevent a bioaccumulation of such metals with aging.

Optionally, a dietary supplement such as those disclosed in co-owned U.S. patent application Ser. No. 10/123,576, can be used to assist the body and support the chelation and transport of the heavy metals from the central nervous system and vascular system into the fecal excretion pathway, as well as other excretion pathways. The dietary supplement may include glutathione and/or metallothionine, alpha lipoic acid, cilantro extract, and/or a precursor such as glycine, cysteine, N-acetylcysteine or methionine, preferably glycine or methionine, more preferably methionine.

A fiber additive in accordance with one embodiment of the present invention comprises a blend of fibers, preferably containing apple fiber, beet fiber, wheat fiber and inulin. This blend of fibers can then be incorporated with a carrier to form a fiber supplement, or be blended with other non-fiber substances to form a dietary supplement containing the fiber blend. Preferably, the fiber blend is incorporated with a food or beverage, and more preferably, the fiber blend is incorporated with a confectionary base. If formulated into a dietary supplement, this may contain many ingredients, among which may be included alpha lipoic acid (thiooctic acid), cilantro (coriander) extract, a bioflavonoid such as quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside), citrus bioflavonoid complex and lemon bioflavonoid complex, among others, and minerals, such as calcium, magnesium, zinc, molybdenum, manganese, chromium, boron, copper, iron, selenium, vanadium and mixtures thereof.

The inclusion of such minerals in a dietary supplement assists the body in replacing the captured heavy metal species with useful minerals.

The fiber supplement may further comprise at least one vitamin selected from the group consisting of thiamine (vitamin B1), vitamin B6, folic acid, vitamin B12, vitamin A, vitamin E, and vitamin C. The roles of antioxidants are well known. The inclusion of vitamins and antioxidants assists the body in repairing tissues on a molecular level and preventing further damage by heavy metal incursions.

The present invention recognizes that heavy metals are present in the natural environment (air, earth, water) and in a host of consumer products. The metals absorbed by the body in trace amounts over the short term accumulate to the eventual detriment of the individual's health, unless the diet provides the individual with the suitable natural components or materials to capture and eject the metal, or allow the body's natural elimination processes to expunge the heavy metals from the body. The fiber supplements/additives of the present invention provide a relatively safe means, using natural ingredients with little/few or no side effects, by which users may cleanse themselves of heavy metals and limit further bio-accumulation of heavy metals going forward. Thus the invention is both a restorative treatment and prophylaxis to limit detrimental effects from continued exposure to trace quantities of such heavy metals.

### Definitions

The term “heavy metal species,” “heavy metal,” “heavy metal atom,” and “heavy metal ion” are used interchangeably herein to designate atoms and cations of those metals which are essentially considered toxic to human beings. Such toxic metals generally do not occur naturally in any significant quantities in human beings and when present in elevated quantities are likely to result in impairment of normal human functioning. Such impairment may affect short term and long term memory, linguistic abilities, social skills, motor skills, cognition and other basic capabilities. Heavy metals typically include mercury, lead, nickel, arsenic, cadmium, aluminum, some species of chromium, and tin with implications for the different heavy metals in different conditions, disease states and symptomology.

### DETAILED DESCRIPTION OF THE INVENTION

The fiber supplements described herein are best suited to long term use and are preferably to be taken regularly, at periodic intervals. The supplements are administrable from once up to six times daily for supplementing and bolstering the body's natural defenses against heavy metals. A preferred schedule is once or twice daily. However, higher rates of consumption are certainly acceptable. As the consumption rate increases, the amounts of the individual components should be decreased.

The compositions described herein are intended as additions to normal diet and should not be considered as substitutes for proper nutrition. It is recommended that the supplements be taken at mealtime to take advantage of the full panoply of nutritive constituents of traditional foods. Used in this way, the compositions described herein are best able to promote and supplement the natural cleansing mechanisms of the body and to assist in the removal of heavy metals.

For assisting the natural metabolic processes of the body in cleansing the intestinal tract of heavy metals, a fiber supplement includes at least one bulk fiber, preferably a soluble fiber, selected from the group consisting of inulin, psyllium, cellulose fiber, lignan, xyloglucan, gums, galactomannan, algal polysaccharide, and sulfated galactans in combination with a metal binding fiber, preferably at least one pectin, such as apple or beet pectin, more preferably both apple and beet pectin, or a water soluble or insoluble oat or wheat bran fiber, or combinations thereof. Note that some sulfated galactans may also be used as a binding fiber in certain embodiments. Apple pectin and beet pectin are known to have the capability of capturing and removing various heavy metal species from the body. Water soluble and water insoluble wheat and oat bran fiber have similar heavy metal binding capability, whereas inulin, psyllium and cellulose are more broad spectrum binding fibers, for supporting the removal of the metal binding fiber with the captured metal species from the body. The combination of these fibers is particularly suited for removing such heavy metals from the body in a safe and effective manner.

Optional ingredients in the fiber supplement may be selected from the group consisting of citrus pectins, such as orange, lemon or lime pectins, chitosan, polyphosphate and combinations thereof.

Preferred fiber blend compositions that may be used as food additives, either in the mass manufacture of foods, or added during individual food preparation, according to the present invention comprise at least about 50% by weight of bulk fiber and no greater than about 50% by weight of a binding fiber, with a preferred composition comprising at least about ⅔ by weight bulk fiber and no more than about ⅓ by weight of at least one metal binding pectin or bran fiber, preferably, at least one pectin and at least one bran fiber. Preferred fiber blend compositions comprise at least about 50% by weight, more preferably at least about 50-80% by weight of a bulk fiber, preferably, inulin, psyllium or cellulose, more preferably, inulin or a mixture of inulin and psyllium and at least about 50% by weight of a mixture of at least one pectin selected from the group consisting of apple pectin, beet pectin and mixtures thereof, and/or optionally, at least one water soluble wheat and/or oat bran fiber and/or a water insoluble wheat bran fiber.

Glutathione or metallothionine support the body in acquiring and moving a heavy metal away from the central nervous system out into an excretion pathway. However, glutathione and metallothionine are generally broken down during the digestive process by digestive enzymes and relatively little of these agents may be delivered efficiently orally. Accordingly, to provide effective amounts of such compounds, the fiber supplement preferably incorporates a precursor used by the body to generate glutathione or metallothionine. Thus, the fiber supplement may include precursors such as glycine, cysteine, n-acetyl cysteine, S-adenosyl methionine and/or methionine or other amino acid, preferably, glycine and/or methionine.

In certain respects, the amounts of the individual fibers used in a fiber supplement as contemplated herein may be relatively small. Generally, about one to two or more grams of the fiber supplement may be administered per day in one or more tablets, capsules, granules or other means, containing about 1% by weight of each of wheat fiber, apple fiber, and beet fiber, and about 12% inulin, the balance of about 85% by weight being the carrier. For example, if formulated for delivery by way of a confectionary base, such as in a gummy candy, the balance would be sucrose, water, gelatin and various color and flavor ingredients. Generally, the blended fibers may comprise from 10 to 30% by weight or more of the product administered, though if formulated with a non-confectionary food or beverage, the amount may more likely be in the range of about 1 to 10%, the proportions of food consumed being usually larger than that of a fiber or dietary supplement.

The fiber blend is administered daily in a single serving or in multiple fractional servings of about 0.05 to about 10 milligrams per kilogram of body weight, more preferably about 0.1 to about 3.5 milligrams per kilogram of body weight, even more preferably about 0.5 to about 2.5 milligrams per kilogram of body weight. Amounts in the lower weight range are preferred where the rate of supplement consumption is high, for instance, five or six times daily, and/or where the purpose of consumption is prophylactic, i.e., to remove the heavy metals as they are acquired, rather than to remove heavy metals which have accumulated over a long period. Conversely, amounts in the higher weight range are preferred where the rate of supplement consumption is low, for instance, one or two times daily, and/or where the purpose of consumption is to cleanse the body of accumulations of heavy metals incurred over a long period or from exposure to unusually high concentrations of the toxic substances. Such a high concentration may occur, for example, when a person has lived for a substantial period near, or in a waste runoff region of, a manufacturing plant using or producing heavy metals.

Another, optional active component of a fiber supplement for assisting the body's natural cleansing mechanisms for removing toxic metals is a vitamin or antioxidant taken from the group consisting of thiamine, vitamin B6, folic acid, vitamin B12, vitamin A, vitamin E, and vitamin C. The roles of vitamins and antioxidants are well known. The inclusion of vitamins and/or antioxidants assists the body in repairing tissues on a molecular level and preventing further damage by heavy metal incursions. These vitamins and/or antioxidants may be included in any desirable combination and in amounts customary in the trade.

A fiber supplement for assisting the body in cleansing itself of toxic metals has at least one fiber selected from the group consisting of apple pectin, beet pectin, water soluble oat or wheat bran, water insoluble wheat bran or mixtures, thereof, and optionally, at least one bulk fiber.

Compositions according to the present invention may be formulated for delivery to a mammal, preferably a human. The compounds of this invention are preferably incorporated into formulations for oral administration, and be in forms suitable for oral administration such as tablets, granules, powders, coated tablets, hard capsules, soft capsules, liquids, suspensions and gels.

One particular novel form of administration is by inclusion in confectionery items. In particular, the presence of heavy metals is not limited to adults and in fact is likely most pronounced in children who have been exposed to such heavy metals, by vaccination, ingestion of lead paints, etc. While the above compositions can be administered by way of tablets or capsules, for example, such forms are not well received by children. Further the compositions of the invention are best administered over time to allow the body's natural mechanisms to remove such substances from the system, and long term consistent compliance, with children in particular, is difficult to achieve if the form of administration is resisted.

Consequently, the fiber compositions of the present invention may be formulated for oral administration as a component of a confectionery, such as being incorporated into soft gummy candy (i.e. gummy bears, worms, etc.), hard candy, gum balls, or chocolate, among others. The fiber blend preferably contains one or more soluble fibers, that is, noncellulosic polysaccharides such as pectins, gums, and mucilage found in fruits, oats, barley and legumes.

In a preferred embodiment, the fiber blend is combined and integrated into a gummy candy formulation, to produce gummy candies, each of which delivers all or a portion of the proper dosage of the fiber ingredients. Preferably these are prepared to contain, for example 1/20th to 1/5th, preferably about 1/10th the daily adult dose per candy piece, such that the weight appropriate amount can be administered to a child. For example while a 250 lb adult might need to consume 10 gummy candies, a 50 lb child may need to consume only 2 or 3, and in this way the appropriate amount, based on weight can easily be determined and provided.

Further, the various components of the inventive formulation can be segregated into different gummy candies or chocolates, to allow further tailoring to individual needs. Thus the fiber blend could be in one gummy, various vitamins and replacement minerals in another, various precursors and/or minerals as discussed above in another, etc, so that a child can eat 4-6 pieces per day and receive the full compliment of fibers, natural chelators, antioxidants, and also mineral replacement. Use of such a delivery system may be very important to achieving long term support of the body's natural system for eliminating contaminants and heavy metals, and to actively prevent further bioaccumulation.

Fiber supplements comprise the above-described components in an effective amount for removing heavy metals from a mammal, especially including a human. One of ordinary skill in the art will recognize that an effective amount of one of more components to be included in the present compositions will vary with the conditions to be resolved and the heavy metal which is to be removed from the individual, as well as the pharmacokinetics of the agent used, and the condition of the patient (animal or human) treated.

The preferred route of administration is by an oral route. Compositions according to the present invention are formulated preferably in admixture with an acceptable carrier, as well as being integrated with confectioneries, food or beverages.

Adjuvants normally used in formulating nutritional supplements may be used in formulating the present invention as carriers, such as syrup, gum Arabic, gelatin, sorbitol, polyvinyl pyrrolidone, magnesium stearate, talc, polyethylene glycol, silica, lactose, sucrose, corn starch, calcium phosphate, starch, carboxymethyl cellulose, water, ethanol, glycerol, mannitol, among others. Optional ingredients such as coloring agents, flavors, dissolution acids, suspending agents, dispensing agents, etc., may also be used. The composition may be formulated to provide delayed or controlled release, using enteric coatings, micro-encapsulation or other techniques known in the art. Of course, for the confectionery form of the invention, any of the components typically used to produce such confectionery items may be used as a base or carrier for delivering the compounds of the invention, adapted, if necessary to avoid detrimental interactions during production.

An effective amount of the composition represents an amount necessary to remove, prevent or limit further bio-accumulation of a heavy metal in the body. The fiber blends described are effective over a wide range of dosages, and it is understood that the dosage may vary based on the symptom to be treated, its severity, the age, weight and response of the individual person, and the chosen route of administration.

Treating in accordance with the present invention may include prophylaxis of heavy metal accumulation, or of a specific symptom, amelioration, elimination, or attenuation of the condition or symptom related to heavy metal toxicity due especially to low level exposure to such heavy metals in the environment.

### EXAMPLES

The following exemplary formulations set forth active ingredients of fiber supplements for assisting the body to carry out natural cleansing processes to rid itself of heavy metal interlopers. Accordingly, each formulation may be used with fillers, buffers, binders, etc., normally found in dietary supplements. Preferably, the non-active ingredients are selected on the basis of their known biocompatibility with the human organism. For instance, possible allergenic substances are to be preferably avoided. Where the compositions are intended for use in children, flavors and food ingredients may be added. Alternatively, the examples below may be implemented as additives to candy, cookies, ice creams and other foods of interest to children. In that event, the amounts of the active ingredients listed in the below examples are selected with due consideration to the amounts of the foods children might be expected to consume in a given (daily) period.

The compositions may be formulated through traditional pharmaceutical compounding procedures and other procedures which are well known in the art. The compositions may be produced in tablet form, gums, oral suspensions, capsules including hard and soft gelatin capsules, among others.

In the examples below, a slash mark (“/”) means that the compounds on opposite sides may be included alternatively in the amount indicated or together in the same weight amount. Note that the individual components are generally weighed out and thoroughly mixed, either as solids or liquids.

### Example 1

| | |
|---|---|
| Inulin | 90 mg |
| Apple Pectin | 10 mg |

### Example 2

| | |
|---|---|
| Inulin | 90 mg |
| Apple pectin | 10 mg |
| Wheat Fiber | 10 mg |

### Example 3

| | | |
|---|---|---|
| Inulin | 125 | mg |
| Apple pectin | 20 | mg |
| Glycine/methionine | 75 | mg |

### Example 4

| | | |
|---|---|---|
| Apple pectin | 25 | mg |
| Beet Pectin | 25 | mg |
| Glycine/methionine | 400 | mg |

### Example 5

| | | |
|---|---|---|
| Apple pectin | 25 | mg |
| Inulin | 100 | mg |
| Beet pectin | 25 | mg |
| Quercetin | 75 | mg |
| Glycine/methionine | 75 | mg |
| Calcium | 100 | mg |
| Magnesium | 50 | mg |
| Zinc | 10 | mg |

### Example 6

| | | |
|---|---|---|
| Apple pectin | 20 | mg |
| Wheat bran fiber | 20 | mg |
| Beet pectin | 20 | mg |
| Inulin | 200 | mg |
| Psyliium | 20 | mg |
| Quercetin | 175 | mg |
| Glycine/methionine | 400 | mg |
| Thiamine | 50 | mg |
| Vitamin B6 | 50 | mg |
| Folic acid | 50 | mg |
| Vitamin B12 | 50 | mg |
| Vitamin A | 50 | mg |
| Vitamin E | 50 | mg |
| Vitamin C | 50 | mg |

| Blend Ingredients | Wt % |
|---|---|
| Apple fiber | 0-30 |
| Beet Fiber | 0-30 |
| Inulin | 1-70 |
| Wheat Fiber | 1-30 |

To any of these examples may be added from 5 to 500 milligrams of a cilantro extract, preferably about 15 to 300 milligrams. Similarly, ginkgo biloba may be used as a substitute for quercetin and/or rutin, in equivalent amounts to those compounds. Alpha lipoic acid may also be used.

### Confectionery Formulations

The following are examples of gummy formulations that may be used for ease of oral administration of the compositions of the invention. The following is illustrative of various ranges for each ingredient, one or more of which, in any combination, can be incorporated into the confectionery formulation.

| Ingredient | Wt (g) |
|---|---|
| Inulin | 36.9 (range 25.0 to 50.0) |
| Beet Fiber | 3.1 (range 2.0 to 8.0) |
| Apple Fiber | 3.1 (range 2.0 to 8.0) |
| Wheat Fiber | 3.1 (range 2.0 to 8.0) |
| *Citric Acid Solution (10%) | 2.8 (range 0-10) |
| *Color | 0.5 |
| *Sucrose | 141.9 |
| *Water | 80.9 and 35.5 |
| *42DE/A | 72.4 |
| *Gelatin | 17.7 |
| *Flavor | 2.13 |

*base confectionery ingredients (chocolate or another confectionary base can be substituted therefore)

400 g produces approximately 200, 2 g gummy candies, with approximately 4 gummies per serving. The base for confectionery formulation may vary but typically includes sucrose, water and gelatin, but many other confectionery bases may be used. For example, chocolate could be substituted to produce individual chocolates, at the same weight ratios as described above.

Generally the confectionery base is the major weight component, with a formulated blend of ingredients integrated therewith. In other words, to a confectionery base may be included from 2 to 20% by weight of the inventive formulations. For example, each of examples 1-6 can be incorporated with a confectionery base, at from 2 to 20% by weight. Thus, various bases, such as hard candy, soft candy, chocolates, chewable or powdered confectioneries may be formulated to deliver the formulations of the invention.

Using the fiber supplement of the invention limits the accumulation of heavy metals in the body, promotes removal of heavy metals previously accumulated in the body and alleviates the numerous symptoms and degenerative or neurocognitive conditions associated with heavy metal toxicity, as well as assists in reducing hyperactivity, chemical sensitivity, allergies fatigue, etc.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A fiber composition for assisting natural body functions in cleansing the body of heavy metals comprising:
at least one bulk fiber selected from the group consisting of inulin, psyllium, cellulose fiber, lignan, xyloglucan, gums, galactomannan, algal polysaccharide, sulfated galactans and mixtures thereof, in combination with at least one metal binding fiber selected from the group consisting of a metal binding pectin, a metal binding bran fiber, or combinations thereof.

2. The fiber composition of claim 1 further comprising a compound selected from the group consisting of glutathione, metallothionine, glycine, methionine and combinations thereof.

3. The composition of claim 1 further comprising a mineral selected from the group consisting of calcium, magnesium, zinc and mixtures thereof.

4. The composition of claim 1 further comprising at least one vitamin or antioxidant selected from the group consisting of thiamine, vitamin B6, folic acid, vitamin B12, vitamin A, vitamin E, vitamin C and combinations thereof.

5. The composition of claim 1 further comprising a bioflavonoid selected from the group consisting of quercetin, rutin, rhametin, fisetin, quercitin, hyperosid, kaempferol, robinin and mixtures thereof.

6. The composition of claim 1 further comprising a carrier, more preferably a confectionary carrier selected from the group consisting of soft candy, hard candy, chocolate and chewing gum.

7. The composition of claim 1 further comprising an additive from the group consisting of cilantro, coriander or extracts thereof, alpha lipoic acid, ginkgo biloba or an extract thereof, and combinations thereof.

8. The composition of claim 1 wherein the fiber composition contains inulin present in an amount of about 5 mg to about 500 mg.

9. The composition of claim 6 wherein the confectionery carrier comprises from 80-90% by weight of the fiber composition.

10. The fiber composition of claim 1 wherein the composition contains from 1.0 to 30% wt inulin, from 0.1 to 10% apple fiber, from 0.1 to 10% wheat fiber and from 0.1 to 10% beet fiber.

11. The composition according to claim 1 wherein said bulk fiber is inulin or a mixture of inulin and psyllium and said metal binding pectin is apple or beet pectin and said metal binding bran fiber is water soluble wheat or oat bran or water insoluble wheat bran.

12. The composition according to claim 1 wherein said bulk fiber is inulin and said metal binding fiber is a mixture of apple and beet pectin.

13. The composition according to claim 12 wherein said metal binding fiber further includes water soluble wheat bran fiber.

14. The composition according to claim 12 wherein said metal binding fiber further includes water insoluble wheat bran fiber.

15. The composition according to claim 12 wherein said metal binding fiber includes water soluble wheat bran fiber, water insoluble wheat bran fiber and optionally, water soluble oat bran fiber.

16. The composition according to claim 1 comprising at least about 50% by weight of bulk fiber and no greater than about 50% by weight of a binding fiber.

17. The composition of claim 1 comprising at least about ⅔ by weight bulk fiber and no more than about ⅓ by weight of at least one metal binding pectin or bran fiber, preferably, at least one pectin and at least one bran fiber.

18. The composition according to claim 1 comprising at least about 50% by weight, more preferably at least about 75-80% by weight of a bulk fiber, preferably, inulin, psyllium or cellulose, more preferably, inulin or a mixture of inulin and psyllium and at least about 50% by weight of a mixture of at least one pectin selected from the group consisting of apple pectin, beet pectin and mixtures thereof, and optionally, at least one water soluble wheat or oat bran fiber or a water insoluble wheat bran fiber.

19. The composition of claim 1 further comprising an ingredient selected from the group consisting of citrus pectins, such as orange, lemon or lime pectins, chitosan, polyphosphate and combinations thereof.

20. A method of removing heavy metals from the gastrointestinal tract of a mammal comprising administering to said mammal a composition containing at least one bulk fiber selected from the group consisting of inulin, psyllium, cellulose fiber, lignan, xyloglucan, gums, galactomannan, algal polysaccharide, sulfated galactans and mixtures thereof, in combination with at least one metal binding fiber selected from the group consisting of a metal binding pectin, a metal binding bran fiber, or combinations thereof.

21. The method of claim 20 further comprising administering the composition from once up to six times daily.

22. The method of claim 20 further comprising administering the composition at from about 0.05 to about 10 milligrams per kilogram of body weight.

23. The method of claim 20 further comprising administering the composition at from about 0.1 to about 3.5 milligrams per kilogram of body weight.

24. The method of claim 20 further comprising administering the composition at from about 0.5 to about 2.5 milligrams per kilogram of body weight.

25. The method of claim 20 wherein the bulk fiber is inulin or a mixture of inulin and psyllium and said metal binding pectin is apple or beet pectin and said metal binding bran fiber is water soluble wheat or oat bran or water insoluble wheat bran.
